(19) United States
(12) Patent Application Publication
(10) Pub. No.: US 2026/0043780 A1
(43) Pub. Date: Feb. 12, 2026

(54) REAGENT SET

(71) Applicant: Endress+Hauser Conducta GmbH+Co. KG, Gerlingen (DE)

(72) Inventors: Hui Huang, Suzhou (CN); Junting Zhang, Suzhou (CN); Ruixin Zhao, Lishui Zhejiang (CN)

(21) Appl. No.: 19/294,738

(22) Filed: Aug. 8, 2025

(30) Foreign Application Priority Data
Aug. 9, 2024 (CN) 202411090169.0

(51) Int. Cl. G01N 33/18; G01N 1/38; G01N 21/31; G01N 31/16

(52) U.S. Cl. CPC G01N 33/1806; G01N 1/38; G01N 21/31; G01N 31/16

(57) ABSTRACT

The present disclosure relates to a reagent set for measuring a chemical oxygen demand in a liquid medium, the reagent set including a first reagent, wherein the first reagent is an acidic solution of mercuric sulfate; a second reagent, wherein the second reagent is an acidic solution comprising potassium dichromate; and a third reagent, wherein the third reagent is an acidic solution, characterized in that at least the second reagent or at least the third reagent further includes potassium aluminum sulfate.

The present application is related to and claims the priority benefit of Chinese Patent Application No. 202411090169.0, filed on Aug. 9, 2024, the entire contents of which are incorporated herein by reference.

### TECHNICAL FIELD

The present disclosure relates to a reagent set, in particular to a reagent set for measuring a chemical oxygen demand (COD) in a liquid medium. The present disclosure further relates to an analysis device and method for measuring a COD using the reagent set.

### BACKGROUND

Chemical oxygen demand (COD) reflects the degree of pollution by reducing substances in water, and this indicator is also one comprehensive indicator of the relative content of organic matter.

To measure the amount of organic matter in water samples, a dichromate method is mainly used in the prior art. The term chemical oxygen demand (COD) refers to, when a liquid medium sample (such as a water sample) is oxidized by potassium dichromate (K₂Cr₂O₇) under specific conditions, the mass concentration, usually expressed in milligrams per liter (mg/L), of corresponding oxygen (O₂) consumed by dissolved substances and suspended matter in the sample. A chemical oxygen demand (COD) test can be used to quantify the amount of organic matter in water. The principle of the dichromate method is described in the National Environmental Protection Standard of the People's Republic of China HJ828-2017, which is: adding a known amount of a potassium dichromate solution to a sample, and using silver sulfate (Ag₂SO₄) as a catalyst in a strong acidic medium; after boiling reflux, using 1,10-phenanathroline monohydrate as an indicator and ammonium ferrous sulfate ([(NH₄)₂Fe(SO₄)₂·6H₂O]) to titrate the unreduced potassium dichromate in the water sample, and calculating the mass concentration of consumed oxygen from the amount of potassium dichromate consumed. Under acidic potassium dichromate conditions, aromatic hydrocarbons and pyridine are less likely to be oxidized and have low oxidation rates. Under the catalysis of silver sulfate, straight-chain aliphatic compounds can be effectively oxidized.

The interference of inorganic matter coexisting in the water samples with the measurements of the chemical oxygen demand has always been a difficult problem to solve. A main interfering substance in the above method is chlorides. High concentrations of chlorides can react with the potassium dichromate (K₂Cr₂O₇) and render the chemical oxygen demand (COD) of the sample overly high. The reaction of the chlorides with the potassium dichromate follows the equation:

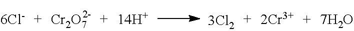

The interference of the chlorides in the sample can be eliminated by adding a mercuric sulfate (HgSO₄) solution. After reflux, chloride ions can combine with mercuric sulfate to form soluble chloromercuric complexes. However, this method is not applicable to the measurement of chemical oxygen demand in water containing chlorides at a concentration greater than 1,000 mg/L. Samples exceeding that concentration need to be diluted before measurement.

The Chinese invention patent with the publication number CN 104515769 A discloses a method for rapid measurement of a COD in high-chloride wastewater, wherein a sample needs to be diluted so that the concentration of chlorides is less than 2,000 mg/L.

The Chinese invention patent with the publication number CN 108956509 A discloses a reagent for rapidly measuring a COD of high-chlorine wastewater and a testing method using same, wherein the concentration of chlorides needs to be lower than 4,000 mg/L, otherwise a sample needs to be diluted.

The European invention patent with the publication number EP 3591395 A1 discloses a method for measuring a chemical oxygen demand (COD) of a high-chlorine sample, the method using a prefabricated reagent and a photometer. The concentration of chlorides in the sample can be between 1,500 mg/L and 20,000 mg/L. If the concentration of chlorides in the sample exceeds 20,000 mg/L, the sample needs to be diluted.

### SUMMARY

In order to overcome the defects of the difficulty in directly measuring a COD value in high-chloride wastewater in the prior art, the present disclosure provides a reagent set for measuring a COD and an analysis device and method for measuring a COD using the reagent set, an analysis device, and a method for measuring a chemical oxygen demand (COD) in a high-chloride liquid medium.

The reagent set used by the present disclosure to solve the technical problem comprises:
a first reagent, wherein the first reagent is an acidic solution of mercuric sulfate (HgSO₄);
a second reagent, wherein the second reagent is an acidic solution comprising potassium dichromate (K₂Cr₂O₇); and
a third reagent, wherein the third reagent is an acidic solution, characterized in that at least the second reagent or at least the third reagent further comprises potassium aluminum sulfate (KAl(SO₄)₂·12H₂O).

In the foregoing reagent set, the function of the mercuric sulfate (HgSO₄) is to eliminate the interference from chlorides in the liquid medium. The potassium dichromate (K₂Cr₂O₇) is a strong oxidant in an acidic environment, and the function thereof is to oxidize organic matter in the liquid medium. The potassium aluminum sulfate (KAl(SO₄)₂·12H₂O) serves as a catalyst. The potassium aluminum sulfate is also known as potassium alum, potassium aluminum alum, potassium alum, white alum, raw alum, alunite, mica alum, and potassium aluminum sulfate dodecahydrate, and is also simply referred to as “alum”. Alum (potassium aluminum sulfate) crystals are colorless or white octahedral crystals that are easily soluble in water, and the crystal structure thereof is a heteropolar trigonal crystal system. An aqueous solution thereof is safe, non-toxic, colorless, and acidic. Potassium aluminum sulfate also has the advantages of being cheap and easily available. When silver sulfate (Ag₂SO₄) is used as a catalyst and the liquid medium contains a high concentration of chlorine, silver ions easily react with chloride ions to form silver chloride (AgCl) precipitates that are not easily soluble in water, reducing the catalytic effect and interfering with test results. Unlike silver sulfate or other silver salts, potassium aluminum sulfate (KAl(SO₄)₂·12H₂O) does not cause precipitation of chloride ions (Cl⁻).

The acidity of the first reagent may be achieved by adding sulfuric acid (H₂SO₄). Similarly, the acidities of the second reagent and the third reagent may also be achieved by adding sulfuric acid (H₂SO₄). It should be understood that the acidity of the foregoing reagent set may also be achieved by adding other strong acids.

In some embodiments, the acidity of the first reagent is achieved by adding 10-15% volume of concentrated sulfuric acid (with a concentration of 96%), the acidity of the second reagent is achieved by adding 10-15% volume of concentrated sulfuric acid (with a concentration of 96%), and the acidity of the third reagent is achieved by adding not less than 90% volume of concentrated sulfuric acid (with a concentration of 95-97%).

In some embodiments, the concentration of the mercuric sulfate (HgSO₄) in the first reagent is 150 g/L to 180 g/L. The mercuric sulfate having a high concentration is to eliminate interference from a high concentration of chlorides.

In some embodiments, the concentration of the potassium dichromate (K₂Cr₂O₇) in the second reagent is 20 g/L to 30 g/L.

In some embodiments, the concentration of the potassium aluminum sulfate (KAl(SO₄)₂·12H₂O) in the second reagent or the third reagent is 5 g/L to 10 g/L.

The present disclosure further solves the technical problem by means of an analysis device. The analysis device comprises:
a. the reagent set according to any one of the above described embodiments;
b. a reaction tank;
c. a treatment system for providing a liquid sample and the first reagent, the second reagent, and the third reagent to the reaction tank;
d. a temperature regulating device at least for regulating the temperatures of the reaction tank and the treated sample contained therein;
e. a measuring sensor for recording at least one measured value of the treated sample contained in the reaction tank, the measured value being related to a measured variable;
f. a control unit for controlling the treatment system; and
g. an evaluation unit for determining a COD value according to the measured value recorded by the measuring sensor.

In some embodiments, the treatment system comprises a sample pump, a valve group, a quantitative feeding unit, and a dispenser, wherein the sample pump, the valve group, and the quantitative feeding unit are used to convey a predetermined amount of the liquid sample from a sample supply to the reaction tank through a flow path, the valve group, the quantitative feeding unit, and the dispenser are further used to supply a predetermined amount of the first reagent, a predetermined amount of the second reagent, and a predetermined amount of the third reagent to the reaction tank, the valve group and the dispenser are further used to supply air to the reaction tank, so that the predetermined amount of the sample and the predetermined amount of the first reagent supplied to the reaction tank are sufficiently mixed via the air, and the predetermined amount of the sample, the predetermined amount of the first reagent, the predetermined amount of the second reagent, and the predetermined amount of the third reagent supplied to the reaction tank are sufficiently mixed via the air, and the control unit is configured to control the sample pump, the valve group, the quantitative feeding unit, and the dispenser.

In some embodiments, the measuring sensor is a photometer, and the measured variable is the absorbance of the treated sample.

In some embodiments, the analysis device further comprises at least one first supply container containing the first reagent, at least one second supply container containing the second reagent, and at least one third supply container containing the third reagent, wherein the first supply container, the second supply container, and the third supply container are respectively connected to the reaction tank through flow paths.

In some embodiments, the analysis device further comprises a transparent shield, the transparent shield being arranged between an operator and the reaction tank and covering at least the reaction tank and the measuring sensor.

In some embodiments, the treatment system further comprises a dilution module for diluting the liquid sample before the liquid sample is provided to the reaction tank.

The present disclosure further solves the technical problem through a method. The method comprises the following steps:
a. providing the analysis device according to any one of the above described embodiments;
b. providing a predetermined amount of a liquid medium sample to a reaction tank by means of a treatment system;
c. adding a predetermined amount of a first reagent to the reaction tank by means of the treatment system, and mixing the predetermined amount of the sample with the predetermined amount of the first reagent;
d. adding a predetermined amount of a second reagent to the reaction tank by means of the treatment system to obtain a predetermined amount of a first analyte;
e. maintaining the first analyte at a first temperature by means of a temperature regulating device, and measuring and recording a first measured value of the first analyte using a measuring sensor;
f. adding a predetermined amount of a third reagent to the reaction tank by means of the treatment system, and mixing the predetermined amount of the sample with the predetermined amount of the first reagent, the predetermined amount of the second reagent, and the predetermined amount of the third reagent to obtain a second analyte;
g. maintaining the second analyte at a second temperature by means of the temperature regulating device, and measuring and recording a second measured value of the second analyte using the measuring sensor; and
h. determining a chemical oxygen demand (COD) of the sample using an evaluation unit according to the first measured value and the second measured value recorded by the measuring sensor.

When a potassium dichromate method is used to measure the COD, the oxidant itself will be reduced from Cr⁶⁺ to Cr³⁺ during the chemical reaction. Relatedly, the color of the solution changes from orange to green. The reduction of chromate and the color change are proportional to the concentration of oxidizable substances in the sample. Therefore, optical quantification of the amount of chromate reduction may be used as an indicator of the chemical oxygen demand of the sample. The aqueous solution of potassium aluminum sulfate is colorless and will not interfere with the optical quantification.

In some embodiments, a specific amount of the sample used is 1 ml to 3 ml.

In some embodiments, a specific amount of the first reagent used is 1 ml to 3 ml.

In some embodiments, a specific amount of the second reagent used is 1 ml to 3 ml.

In some embodiments, a specific amount of the third reagent used is 1 ml to 3 ml.

In some embodiments, in step e), the first temperature is 50° C. to 60° C., and the first analyte is maintained at the first temperature for 3 minutes to 10 minutes before the first measured value is measured.

In some embodiments, in step g), the second temperature is 100° C. to 175° C., and the second analyte is maintained at the second temperature for 3 minutes to 120 minutes before the second measured value is measured.

In some embodiments, the method further comprises flushing the reaction tank and the treatment system with a liquid medium before step b).

A beneficial effect of the present disclosure is that a COD value in high-chlorine wastewater can be directly measured using the reagent set and method provided in the present disclosure without the need to dilute a sample.

As used in the description, “high chlorides” means that the concentration of chlorides in the liquid medium is high. The reagent set, analysis device, and method disclosed in the present disclosure can at least measure a COD value of a liquid medium containing chlorides in an amount up to 20,000 mg/L. The reagent set and method of the present disclosure are not only applicable to liquid media with very high chloride concentrations, but also applicable to liquid media with very low chloride concentrations. It should be understood that the reagent set, analysis device, and method of the present disclosure are at least applicable to measurement of a COD value of a liquid medium with a chloride concentration of 0 mg/L to 30,000 mg/L, inclusive. When the chloride concentration of the liquid medium is higher than 30,000 mg/L, the COD value thereof can be indirectly measured by diluting the liquid medium.

The reagent set, analysis device, and method disclosed in the present disclosure can at least measure a COD value of a liquid medium with a COD value as high as 4,000 mg/L. It should be understood that the reagent set, analysis device, and method of the present disclosure are at least applicable to measurement of a COD value of a liquid medium with a COD value between 0 mg/L and 4,000 mg/L, inclusive.

As used in the description, the “liquid medium” comprises industrial wastewater, domestic sewage, and coastal seawater.

### BRIEF DESCRIPTION OF THE DRAWINGS

The present disclosure will be further described below with reference to the accompanying drawings and embodiments.

FIG. 1 shows Embodiment 4 of an analysis device for measuring a COD value of a liquid medium using a reagent set of the present disclosure;

FIG. 2 shows Embodiment 5 of an analysis device;

FIG. 3 shows Embodiment 6 of an analysis device;

FIG. 4 shows Embodiment 7 of an analysis device.

### DETAILED DESCRIPTION OF PREFERRED EMBODIMENTS

In Embodiment 1, a reagent set for measuring a chemical oxygen demand (COD) of a liquid medium included a first reagent, a second reagent, and a third reagent, where the second reagent was a mixed acidic solution of potassium dichromate (K₂Cr₂O₇) and potassium aluminum sulfate (KAl(SO₄)₂·12H₂O), and the third reagent was concentrated sulfuric acid. The specific components are shown in Table 1.

TABLE 1

| | First reagent | | Second reagent | | | Third reagent |
|---|---|---|---|---|---|---|
| Component | HgSO₄ | H₂SO₄ | K₂Cr₂O₇ | KAl(SO₄)₂•12H₂O | H₂SO₄ | H₂SO₄ |
| Concentration | 150-180 g/L | 10-15% | 20-30 g/L | 5-10 g/L | 10-15% | 100% |

As shown in Table 1, the acidities of the first reagent, the second reagent, and the third reagent were achieved by adding sulfuric acid (H₂SO₄). The acidity of the first reagent was achieved by adding 10-15% volume of concentrated sulfuric acid, the acidity of the second reagent was achieved by adding 10-15% volume of concentrated sulfuric acid (with a concentration of 96%), and the third reagent was 100% volume of concentrated sulfuric acid (with a concentration of 95-97%).

Specifically, for example, to prepare 1000 ml of the first reagent, first, 100 ml to 150 ml of the concentrated sulfuric acid (with a concentration of 96%) and 600 ml to 750 ml of ultrapure water were sufficiently mixed in a beaker, and then 150 g to 180 g of mercuric sulfate (HgSO₄) was added and fully dissolved. After the temperature of the reagent in the beaker returned to room temperature, the reagent was transferred to a volumetric flask, and ultrapure water was injected into the volumetric flask to a 1000 ml mark to obtain the first reagent.

In Embodiment 2, a reagent set for measuring a chemical oxygen demand (COD) of a liquid medium also included a first reagent, a second reagent, and a third reagent, but the components of the second reagent and the third reagent were different from those in Embodiment 1. The second reagent was an acidic solution of potassium dichromate (K₂Cr₂O₇), and the third reagent was an acidic solution of potassium aluminum sulfate (KAl(SO₄)₂·12H₂O). The specific components are shown in Table 2.

TABLE 2

| | First reagent | | Second reagent | | Third reagent | |
|---|---|---|---|---|---|---|
| Component | HgSO₄ | H₂SO₄ | K₂Cr₂O₇ | H₂SO₄ | KAl(SO₄)₂•12H₂O | H₂SO₄ |
| Concentration | 150-180 g/L | 10-15% | 20-30 g/L | 10-15% | 5-10 g/L | ≥90% |

In Embodiment 3, a reagent set for measuring a chemical oxygen demand (COD) of a liquid medium also included a first reagent, a second reagent, and a third reagent, where potassium aluminum sulfate (KAl(SO₄)₂·12H₂O) was present in both the second reagent and the third reagent. The specific components are shown in Table 3.

TABLE 3

| | First reagent | | Second reagent | | | Third reagent | |
|---|---|---|---|---|---|---|---|
| Component | HgSO₄ | H₂SO₄ | K₂Cr₂O₇ | KAl(SO₄)₂•12H₂O | H₂SO₄ | KAl(SO₄)₂•12H₂O | H₂SO₄ |
| Concentration | 150-180 g/L | 10-15% | 20-30 g/L | 5-10 g/L | 10-15% | 5-10 g/L | ≥90% |

As shown in Table 3, Embodiment 3 differed from Embodiment 1 and Embodiment 2 in that the second reagent and the third reagent both included potassium aluminum sulfate (KAl(SO₄)₂·12H₂O). That is, the second reagent was a mixed acidic solution of potassium dichromate (K₂Cr₂O₇) and potassium aluminum sulfate (KAl(SO₄)₂·12H₂O). It should be understood that when the reagent set in Embodiment 3 is used to measure a COD, if the amounts of a sample, the first reagent, the second reagent, and the third reagent used are the same as those in Embodiment 1 or Embodiment 2, the concentrations of the potassium aluminum sulfate (KAl(SO₄)₂·12H₂O) in the second reagent and the third reagent might be appropriately reduced.

In Embodiment 4, an analysis device 100 for measuring a chemical oxygen demand (COD) in a liquid medium included a reaction tank 101, a treatment system 102, a temperature regulating device 107, a measuring sensor 108, a control unit 109, and an evaluation unit 110.

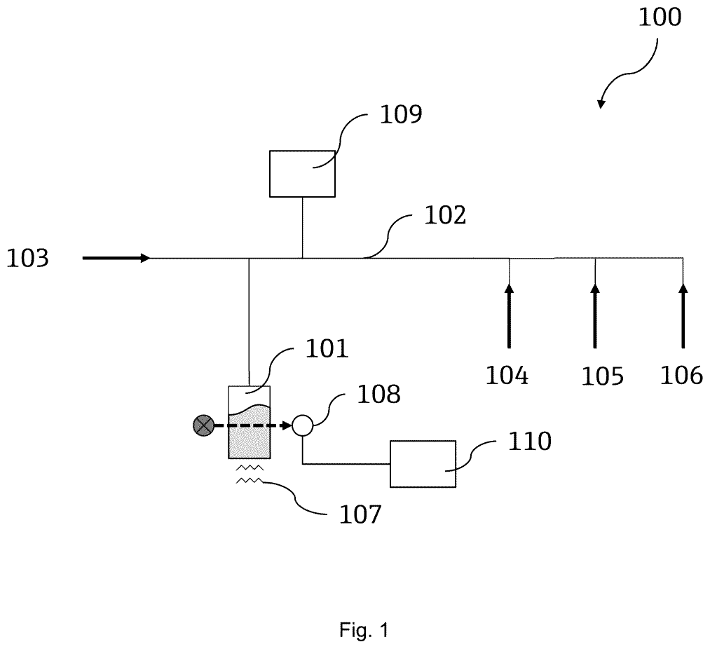

As shown in FIG. 1, first, 1 ml to 3 ml of a liquid medium sample 103 was introduced into the reaction tank 101 of the analysis device 100, and may specifically have been introduced by means of the treatment system 102. Next, 1 ml to 3 ml of a first reagent 104, such as an acidic solution of mercuric sulfate (HgSO₄), was introduced into the reaction tank 101, so that the sample 103 was mixed with the first reagent 104, where the concentration of the mercuric sulfate (HgSO₄) was 150 g/L to 180 g/L. Then, 1 ml to 3 ml of a second reagent 105, such as an acidic solution of potassium dichromate (K₂Cr₂O₇), was introduced into the reaction tank 101, where the concentration of the potassium dichromate (K₂Cr₂O₇) was 20 g/L to 30 g/L. In this way, the mixed liquid in the reaction tank 101 was a first analyte. The reaction tank 101 and the first analyte were maintained by the temperature regulating device 107 at a first temperature, specifically 50° C. to 60° C., and maintained for 3 minutes to 10 minutes, and then a first measured value of the first analyte was measured using the measuring sensor 108. The measuring sensor 108 may be a photometer, and the first measured value was an absorbance value. The purpose of maintaining the first analyte at the first temperature was to eliminate the influence of ambient temperature on the measurement to obtain a stable first measured value.

Then, 1 ml to 3 ml of a third reagent 106, such as an acidic solution of potassium aluminum sulfate (KAl(SO₄)₂·12H₂O), was introduced into the reaction tank 101, where the concentration of the potassium aluminum sulfate (KAl(SO₄)₂·12H₂O) was 5 g/L to 10 g/L. At this time, the sample 103 was sufficiently mixed with the first reagent 104, the second reagent 105, and the third reagent 106 in the reaction tank 101, and the mixed liquid was a second analyte. After the second analyte was obtained, the reaction tank 101 and the second analyte therein were maintained using the temperature regulating device 107 at a second temperature, specifically 100° C. to 175° C., and the reaction was made to continue for 3 minutes to 120 minutes. When the color of the second analyte no longer changed, a second measured value thereof might be measured by the measuring sensor 108. The second measured value might be an absorbance value. The duration of the reaction was related to the COD value of the sample 103. The higher the COD value, the longer the reaction time.

Finally, the chemical oxygen demand (COD) of the sample 103 was determined using the evaluation unit 110 according to the first measured value and the second measured value measured and recorded by the measuring sensor 108.

If necessary, before the liquid medium sample 103 was introduced into the reaction tank 101, the reaction tank 101 and the treatment system 102 might have been flushed with a liquid medium.

Embodiment 5 is an example of using a Liquiline System CA80 COD analysis device 200 of Endress+Hauser to measure a chemical oxygen demand (COD) in a liquid medium. The analysis device 200 included a reaction tank 201, a treatment system 202, a temperature regulating device 207, a measuring sensor 208, a control unit 209, and an evaluation unit 210.

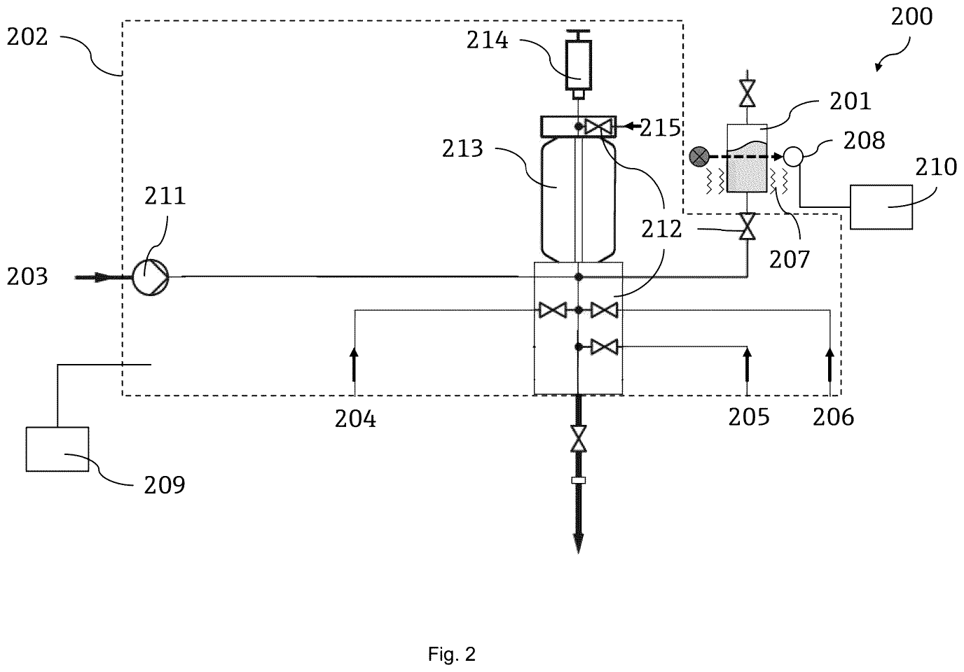

As shown in FIG. 2, a sample 203 was introduced into the reaction tank 201 of the analysis device 200 by means of the treatment system 202. The treatment system 202 further included a sample pump 211, a valve group 212, a quantitative feeding unit 213, and a dispenser 214. The sample 203 was introduced into the analysis device 200 by means of the sample pump 211, and the valve group 212 and the quantitative feeding unit 213 were used to quantify the liquid sample 203 and convey same to the reaction tank 201 through a flow path. The valve group 212, the quantitative feeding unit 213, and the dispenser 214 were further used to supply a predetermined amount of a first reagent 204, a predetermined amount of a second reagent 205, and a predetermined amount of a third reagent 206 to the reaction tank 201.

The valve group 212 and the dispenser 214 were not only used to supply the sample 203 and the reagent set, but also used to supply air 215 to the reaction tank 201, so that the predetermined amount of the sample 203 supplied to the reaction tank 201 and the predetermined amount of mercuric sulfate (HgSO₄) in the first reagent 204 were sufficiently mixed via the air 215, thereby eliminating the interference from chlorides in the liquid medium. Likewise, the predetermined amount of the sample 203, the predetermined amount of the first reagent 204, the predetermined amount of the second reagent 205, and the predetermined amount of the third reagent 206 supplied to the reaction tank 201 might also have been sufficiently mixed via the air 215.

The control unit 209 was configured to control the treatment system 202, which included the sample pump 211, the valve group 212, the quantitative feeding unit 213, and the dispenser 214.

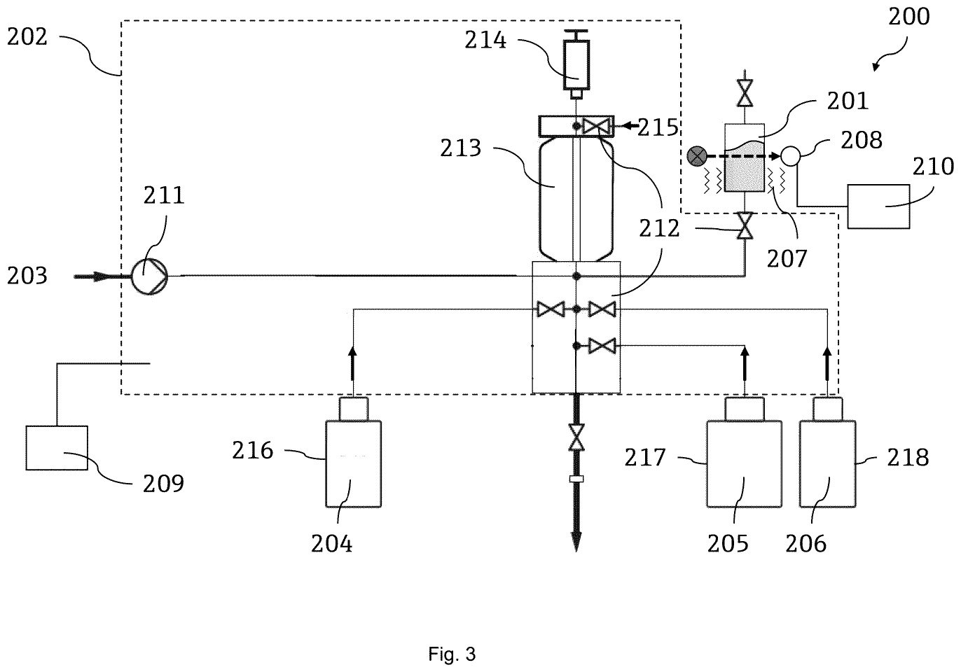

As shown in FIG. 3, the analysis device 200 of Embodiment 6 further included at least one first supply container 216 containing the first reagent 204, at least one second supply container 217 containing the second reagent 205, and at least one third supply container 218 containing the third reagent 206, where the first supply container 216, the second supply container 217, and the third supply container 218 were respectively connected to the reaction tank 201 through flow paths.

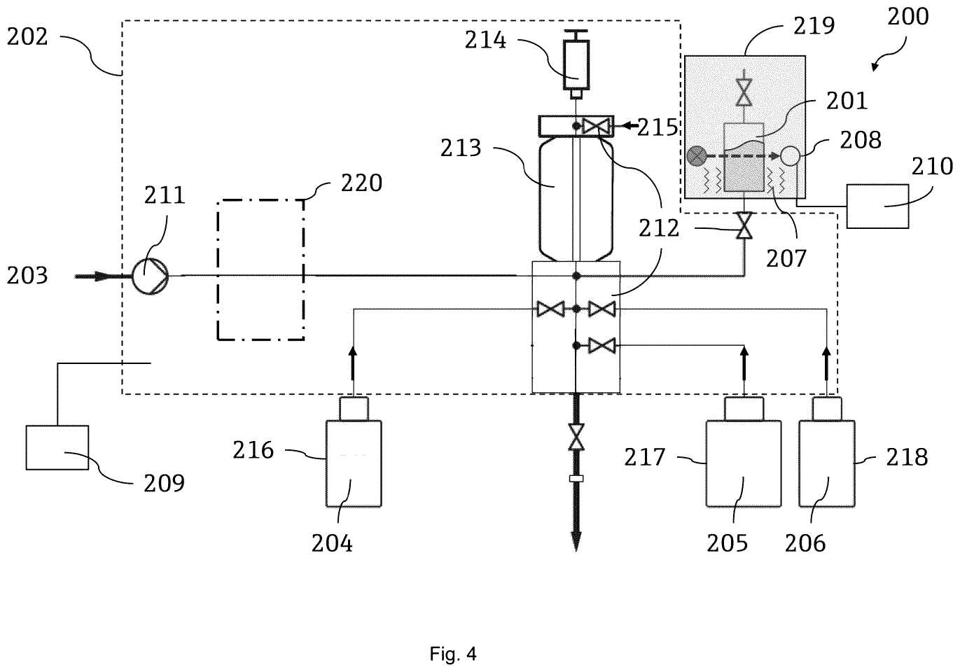

As shown in FIG. 4, the analysis device 200 of Embodiment 7 further included a transparent shield 219. The transparent shield 219 was located between an operator and the reaction tank 201 and covered at least the reaction tank 201 and the measuring sensor 208 to protect the operator. During a measurement process, the environment inside of the reaction tank 201 might have a high temperature and a high pressure. The presence of the transparent shield 219 could effectively protect the operator, for example, when the reaction tank 201 bursts due to improper operation.

The treatment system 202 of the analysis device 200 further included an optional dilution module 220 for diluting the sample 203. The dilution module 220 might further include water for dilution and a valve (not shown), and the control unit 209 was further configured to control the treatment system, which included the dilution module 220. The reagent set, the analysis device 200, and the method disclosed in the present disclosure can directly measure the COD value of the sample 203; therefore, the dilution module 220 is not necessary. However, when the chloride content of the sample 203 exceeds a range that can be directly measured by the analysis device 200, the dilution module 220 may be optionally added to indirectly measure the COD value of the sample 203 containing chloride ions at an overly high concentration.

1. A reagent set for measuring a chemical oxygen demand in a liquid medium, the reagent set comprising:
a first reagent, wherein the first reagent is an acidic solution comprising mercuric sulfate;
a second reagent, wherein the second reagent is an acidic solution comprising potassium dichromate; and
a third reagent, wherein the third reagent is an acidic solution;
wherein at least the second reagent or at least the third reagent further comprises potassium aluminum sulfate.

2. The reagent set according to claim 1, wherein an acidity of the first reagent, an acidity of the second reagent, and an acidity of the third reagent are achieved by adding sulfuric acid, wherein:
the acidity of the first reagent is achieved by adding in the range of 10% to 15% volume of concentrated sulfuric acid;
the acidity of the second reagent is achieved by adding in the range of 10% to 15% volume of concentrated sulfuric acid with a concentration of 96%; and
the acidity of the third reagent is achieved by adding not less than 90% volume of concentrated sulfuric acid with a concentration in the range of 95% to 97%.

3. The reagent set according to claim 1, wherein:
a concentration of the mercuric sulfate in the first reagent is in the range of 150 g/L to 180 g/L.

4. The reagent set according to claim 1, wherein:
a concentration of the potassium dichromate in the second reagent is in the range of 20 g/L to 30 g/L.

5. The reagent set according to claim 1, wherein:
a concentration of the potassium aluminum sulfate in the second reagent or the third reagent is in the range of 5 g/L to 10 g/L.

6. An analysis device for measuring a chemical oxygen demand in a liquid medium, the analysis device comprising:
the reagent set according to claim 1;
a reaction tank;
a treatment system configured to provide a liquid sample and the first reagent, the second reagent, and the third reagent to the reaction tank;
a temperature regulating device at least configured to regulate a temperature of the reaction tank and a temperature of the liquid medium;
a measuring sensor configured to record at least one measured value of the liquid medium contained in the reaction tank, wherein the measured value is related to a measured variable;
a control unit configured to control the treatment system; and
an evaluation unit configured to determine the chemical oxygen demand value according to the measured value recorded by the measuring sensor.

7. The analysis device according to claim 6, wherein the treatment system comprises a sample pump, a valve group, a quantitative feeding unit, and a dispenser, wherein:
the sample pump, the valve group, and the quantitative feeding unit are configured to convey a predetermined amount of the liquid sample from a sample supply to the reaction tank through a flow path;
the valve group, the quantitative feeding unit, and the dispenser are configured to supply a predetermined amount of the first reagent, a predetermined amount of the second reagent, and a predetermined amount of the third reagent to the reaction tank;
the valve group and the dispenser are configured to supply air to the reaction tank, such that the predetermined amount of the sample and the predetermined amount of the first reagent supplied to the reaction tank are sufficiently mixed via the air, and the predetermined amount of the sample, the predetermined amount of the first reagent, the predetermined amount of the second reagent, and the predetermined amount of the third reagent supplied to the reaction tank are sufficiently mixed via the air; and
the control unit is configured to control the sample pump, the valve group, the quantitative feeding unit, and the dispenser.

8. The analysis device according to claim 6, wherein
the measuring sensor is a photometer, and the measured variable is the absorbance of the treated sample.

9. The analysis device according to claim 6, further comprising:
at least one first supply container, at least one second supply container, and at least one third supply container, wherein the at least one first supply container, the at least one second supply container, and the at least one third supply container are respectively configured to contain the first reagent, the second reagent, and the third reagent, and are connected to the reaction tank by flow paths.

10. The analysis device according to claim 6, further comprising a transparent shield, wherein the transparent shield is arranged between an operator and the reaction tank and covers at least the reaction tank and the measuring sensor.

11. The analysis device according to claim 6, wherein
the treatment system further comprises a dilution module for diluting the liquid sample before the liquid sample is provided to the reaction tank.

12. A method for measuring a chemical oxygen demand in a liquid medium, the method comprising the following steps:
providing the analysis device according to claim 6;
providing a predetermined amount of the liquid medium to the reaction tank using the treatment system;
adding a predetermined amount of a first reagent to the reaction tank using the treatment system, and mixing the predetermined amount of the sample with the predetermined amount of the first reagent;
adding a predetermined amount of a second reagent to the reaction tank using the treatment system to obtain a predetermined amount of a first analyte;
maintaining the first analyte at a first temperature using the temperature regulating device, and measuring and recording a first measured value of the first analyte using the measuring sensor;
adding a predetermined amount of a third reagent to the reaction tank using the treatment system and mixing the predetermined amount of the liquid medium with the predetermined amount of the first reagent, the predetermined amount of the second reagent, and the predetermined amount of the third reagent to obtain a second analyte;
maintaining the second analyte at a second temperature using the temperature regulating device, and measuring and recording a second measured value of the second analyte using the measuring sensor; and
determining the chemical oxygen demand of the liquid medium from the first measured value and the second measured value recorded by the measuring sensor using the evaluation unit.

13. The method according to claim 12, wherein
the predetermined amount of the liquid medium is in the range of 1 ml to 3 ml.

14. The method according to claim 12, wherein
the predetermined amount of the first reagent is in the range of 1 ml to 3 ml.

15. The method according to claim 12, wherein
the predetermined amount of the second reagent is in the range of 1 ml to 3 ml.

16. The method according to claim 12, wherein
the predetermined amount of the third reagent is in the range of 1 ml to 3 ml.

17. The method according to claim 12, wherein,
in the step of maintaining the first analyte at a first temperature, the first temperature is in the range of 50° C. to 60° C., and the first analyte is maintained at the first temperature in the range of 3 minutes to 10 minutes before measuring the first measured value.

18. The method according to claim 12, wherein,
in the step of maintaining the second analyte at a second temperature, the second temperature is in the range of 100° C. to 175° C., and the second analyte is maintained at the second temperature in the range of 3 minutes to 120 minutes before measuring the second measured value.

19. The method according to claim 12, further comprising the step of:
flushing the reaction tank and the treatment system with a liquid medium prior to performing the step of providing a predetermined amount of a liquid medium sample.
